(19) United States
(12) Patent Application Publication
(10) Pub. No.: US 2021/0201242 A1
(43) Pub. Date: Jul. 1, 2021

(54) BEHAVIOR IMPROVEMENT SYSTEM AND BEHAVIOR IMPROVEMENT METHOD

(71) Applicant: BE&DO INC., Osaka (JP)

(72) Inventors: Kazume NIWAI, Osaka (JP); Eiichi OKAMOTO, Osaka (JP); Toyoki HASHIMOTO, Osaka (JP); Li ZHENG, Osaka (JP)

(21) Appl. No.: 17/056,722

(22) PCT Filed: May 29, 2019
(86) PCT No.: PCT/JP2019/021382

§ 371 Date: Nov. 18, 2020

(30) Foreign Application Priority Data
Jun. 6, 2018 (JP) 2018-108782

(51) Int. Cl. G06Q 10/06; G16H 10/20

(52) U.S. Cl. CPC G06Q 10/06398; G16H 10/20; G06Q 10/063114

(57) ABSTRACT

A behavior improvement system (200) according to the present invention, which is a behavior analysis system that analyzes a behavior of a user by using a network system enabling information exchange in an organization including two or more users, includes: a goal information setting unit (210) that sets goal information (400); a performance information acquisition unit (220) that acquires behavior performance information (500) and reaction performance information (600); an evaluation information acquisition unit (230) that acquires evaluation information; a model user selection unit (240) that selects a model user serving as a goal from the two or more users based on the evaluation information; and a model user information presentation unit (250) that presents goal information, behavior performance information, and reaction performance information on the model user. Presenting the performance information on the model user serving as the goal allows each user to autonomously work on behavior improvements without relying on a diagnostician, an advisor, and the like.

### FIELD OF THE INVENTION

The present invention relates to a behavior improvement system that prompts a user to work on behavior improvements, and more particularly to a behavior improvement system that, by using a network system for enabling information exchange in an organization including two or more users, prompts the users to work on behavior improvements.

### BACKGROUND OF THE INVENTION

Generally, an organization as exemplified by a company, a school, or the like employs a personnel evaluation system that evaluates the performance of an individual person by judging the degree of achievement of a goal set by the individual person. The personnel evaluation system, of which objectivity and fairness are demanded, has a variety of ingenious ideas incorporated therein. An individual person judges the degree of achievement of a goal set by himself/herself, and additionally a boss or supervisor of the individual person judges the degree of achievement of the goal, too, so that the performance of the individual person can be judged objectively.

This personnel evaluation system is systematized, and adopted by various organizations such as companies, schools, and the like.

Recent years see the prevalence of a system that not only judges the performance of the individual person but also analyzes points to be reconsidered or improved, for helping the individual person's career advancement or enhancement of the future productivity.

For instance, there is a technique relating to a behavior improvement system that contributes to reforming an individual person's behavior by making the individual person aware of his/her behavior (see Patent Literature 1 (PTL 1)). To be specific, with the behavior improvement system disclosed in PTL 1, a member sets an action plan by himself/herself, checks his/her behavior in view of the set action plan, and recognizes a gap between a self-diagnosis and an other's (diagnostician's) diagnosis. It is also possible to encourage behavior improvements with a higher quality based on an advice that an advisor gives under a result of the diagnosis.

### PRIOR ART LITERATURE

### Patent Literature

PTL 1: International Publication No. W02007/063605

### SUMMARY OF THE INVENTION

### Problem to be Solved by the Invention

The behavior improvement system disclosed in PTL 1, however, requires the presence of an appropriate diagnostician and an appropriate advisor, because a person's behavior is diagnosed by another person (diagnostician) and an advice for behavior improvements is given by an advisor. The diagnostician and advisor are burdened with too much responsibilities.

The present invention has been accomplished in view of the foregoing problems. One of the objects of the present invention is to provide a behavior improvement system that presents performance information on a model user serving as a goal, to thereby let each user autonomously work on behavior improvements without relying on a diagnostician, an advisor, and the like.

### Means for Solving the Problem

To attain the object, the present invention at least includes the configurations described below, or performs the procedures described below. While the descriptions below may contain reference signs, etc. given on the drawings for ease of understanding the present invention, the elements of the present invention are not limited to the ones illustrated in the darwings, but should be construed broadly within the scope technically understandable by those skilled in the art.

A behavior improvement system according to an aspect of the present invention is a behavior analysis system that prompts a user to work on behavior improvements by using a network system that enables information exchange in an organization including two or more users, the system including: a goal information setting unit that sets goal information relating to an individual goal of the user; a performance information acquisition unit that acquires behavior performance information and reaction performance information inputted by the user, the behavior performance information relating to a behavior of the user toward the individual goal, the reaction performance information relating to a reaction of the user to a behavior of another user different from the user; an evaluation information acquisition unit that acquires evaluation information on the user; a model user selection unit that selects a model user serving as a goal from the two or more users based on evaluation information acquired by the evaluation information acquisition unit; and a model user information presentation unit that presents goal information, behavior performance information, and reaction performance information on the model user selected by the model user selection unit.

With this configuration, the performance information on the model user serving as the goal is presented, and thereby the behavior improvement system according to the aspect of the present invention allows each user to autonomously work on behavior improvements without relying on a diagnostician, an advisor, and the like.

Preferably, the goal information includes information relating to a result goal that requires a predetermined period before being achieved and information relating to a behavior goal that is necessary in achieving the result goal.

With this configuration in which the goal information includes the result goal and the behavior goal, the behavior improvement system according to the aspect of the present invention allows each user to autonomously work on behavior improvements in a more specific manner.

Preferably, the behavior performance information includes information relating to the degree of achievement of the result goal and behavior goal.

With this configuration, the behavior improvement system according to the aspect of the present invention enables the user to recognize the performance in more detail and to recognize a position relative to the goal, which leads to an increased motivation of the user.

Preferably, the reaction performance information includes information relating to approving and/or encouraging a behavior of another user different from the user.

With this configuration, the behavior improvement system according to the aspect of the present invention is able to recognize the degree of interest in another user, and based on this information, enables each user to autonomously work on behavior improvements.

Preferably, the behavior improvement system further includes a feedback unit that feeds back evaluation information acquired by the evaluation information acquisition unit after the goal information, behavior performance information, and reaction performance information on the model user are presented by the model user information presentation unit.

With this configuration, the behavior improvement system according to the aspect of the present invention is able to select the model user more appropriately.

Preferably, the model user selection unit selects the model user from ones of the two or more users belonging to the same group as the user does.

With this configuration, the behavior improvement system according to the aspect of the present invention enables the user to recognize the goal information, behavior performance information, and reaction performance information on the model user who is familiar to the user, and thus allows the user to work on ideal and realistic behavior improvements.

Preferably, the group is constituted based on commonness of users in terms of at least any of a job category, a job title, an age, an individual goal, or a behavior tendency.

With this configuration, the behavior improvement system according to the aspect of the present invention enables the user to recognize the goal information, behavior performance information, and reaction performance information on the model user who is familiar to the user and is in the same circumstances as the user is in, and thus allows the user to work on more ideal and realistic behavior improvements.

Preferably, the evaluation information is accomplishment information indicating an accomplishment of the user.

With this configuration, the behavior improvement system according to the aspect of the present invention selects the model user based on objective evaluation information, and thus enables the user to work on appropriate behavior improvements.

Preferably, the evaluation information is stress information indicating a stress condition of the user.

With this configuration, the behavior improvement system according to the aspect of the present invention selects the model user based on objective stress information, and thus allows the user to work on behavior improvements in relation to an appropriate stress condition.

Preferably, the behavior improvement system further includes: a point-to-be-improved analysis unit that compares the goal information, behavior performance information, and reaction performance information on the user against the goal information, behavior performance information, and reaction performance information on the model user, and analyzes a point to be improved in the individual goal of the user, the behavior of the user, the reaction of the user to a behavior of another user different from the user; and a recommendation presentation unit that presents a recommendation in accordance with the point to be improved analyzed by the point-to-be-improved analysis unit.

With this configuration, the behavior improvement system according to the aspect of the present invention analyzes a point to be improved of the user, and accordingly presents a recommendation, thus allowing each user to autonomously work on behavior improvements more easily without relying on a diagnostician, an advisor, and the like.

Preferably, the behavior improvement system further includes a correlation analysis unit that analyzes a correlation of behavior performance information and reaction performance information acquired by the performance information acquisition unit with evaluation information acquired by the evaluation information acquisition unit.

With this configuration, the behavior improvement system according to the aspect of the present invention can grasp a factor in an objective result, and thus allows each user to autonomously work on behavior improvements more appropriately.

Preferably, the behavior improvement system further includes a motivation information acquisition unit that provides a questionnaire to the user at a predetermined timing, and based on a result of the user's answering the questionnaire, acquires motivation information indicating a psychological state of the user, wherein the correlation analysis unit analyzes a correlation among the behavior performance information, the reaction performance information, the evaluation information, and the motivation information.

With this configuration, the behavior improvement system according to the aspect of the present invention can grasp a factor in an objective result including the motivation information in more detail, so that autonomously working on behavior improvements in accordance with each individual user can be allowed more appropriately.

Preferably, the predetermined timing comes when the user sets an individual goal, when the user inputs behavior performance information and reaction performance information, or for every fixed period.

With this configuration, the behavior improvement system according to the aspect of the present invention can acquire more accurate information depending on circumstances, and also can grasp a factor in an objective result more accurately, so that autonomously working on behavior improvements in accordance with each individual user can be allowed more appropriately.

Preferably, the behavior improvement system further includes further includes a health information acquisition unit that acquires health information indicating a health state of the user, wherein the correlation analysis unit analyzes a correlation among the behavior performance information, the reaction performance information, the evaluation information, and the health information.

With this configuration, the behavior improvement system according to the aspect of the present invention can grasp a factor in an objective result including the health information in still more detail, so that autonomously working on behavior improvements in accordance with each individual user can be allowed more appropriately.

Processes executed by the behavior improvement system according to the aspect of the present invention can be interpreted as a behavior improvement method that gives a series of processing procedures. The method is provided in the form of a program for causing a computer to execute the series of processing procedures. The program may be installed on the computer by being downloaded from a predetermined server, by being stored in a predetermined server that is accessible via Internet, or by being recorded on a computer-readable recording medium. Function blocks included in the behavior improvement system according to the aspect of the present invention may be partially or entirely implemented as a large-scale integration (LSI) which is an integrated circuit, for example.

### Effect of the Invention

As described above, the present invention can achieve a behavior improvement system that allows each user to autonomously work on behavior improvements without relying on a diagnostician, an advisor, and the like, by presenting performance information on a model user serving as a goal.

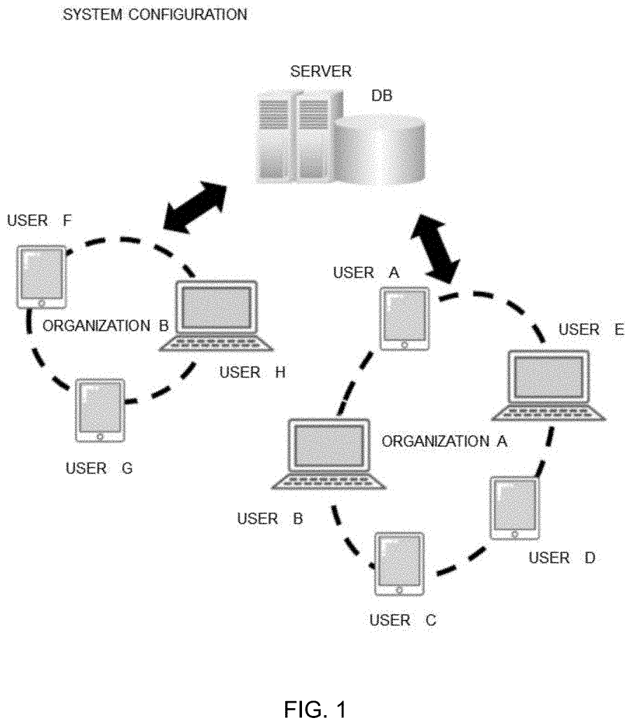

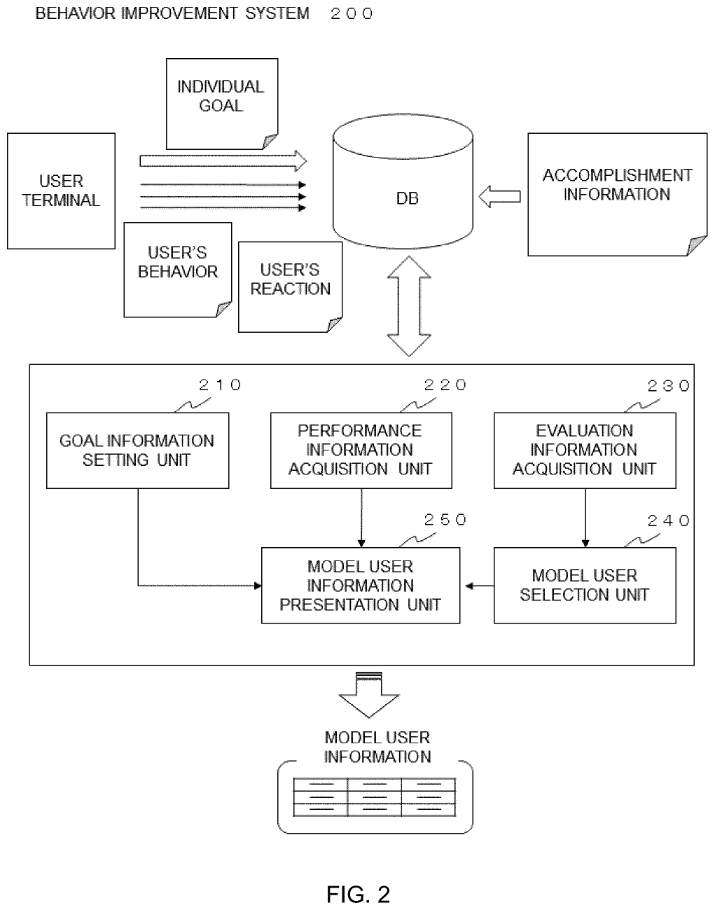

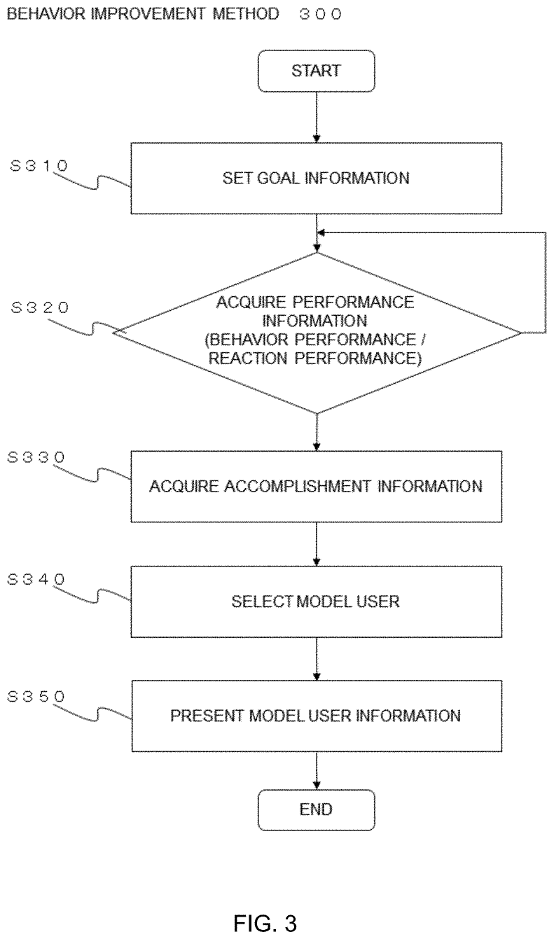

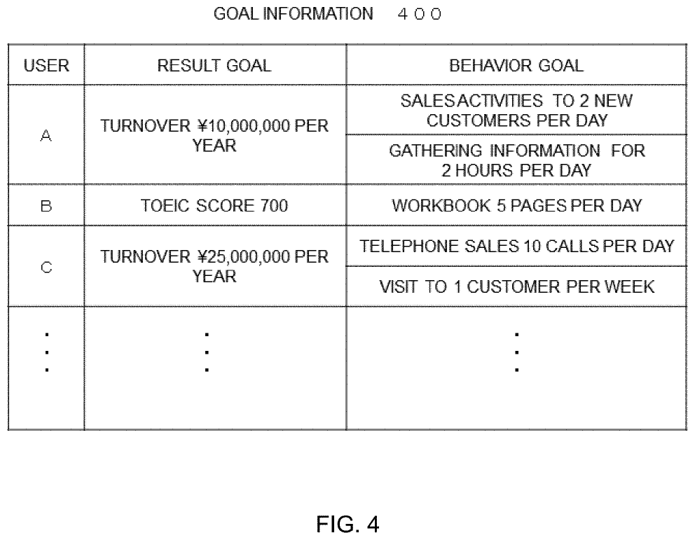

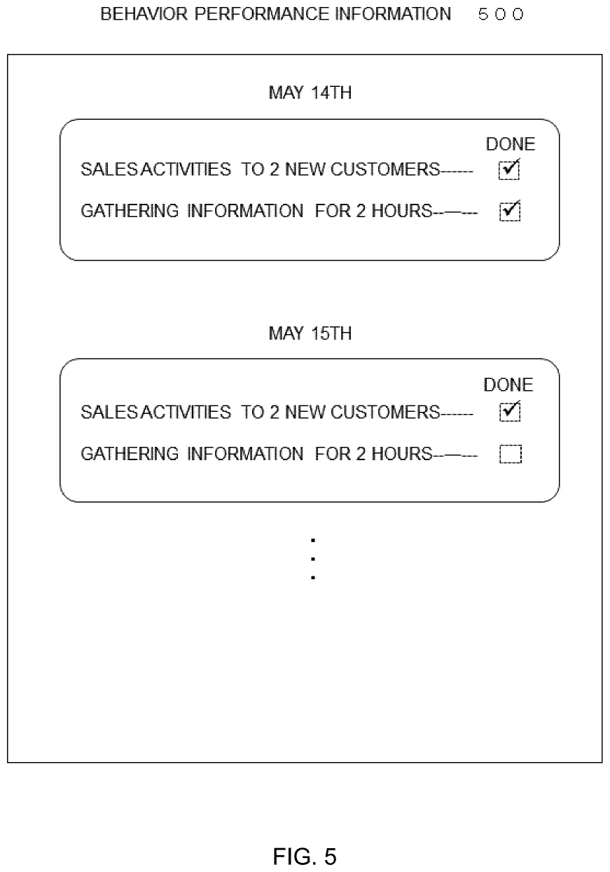

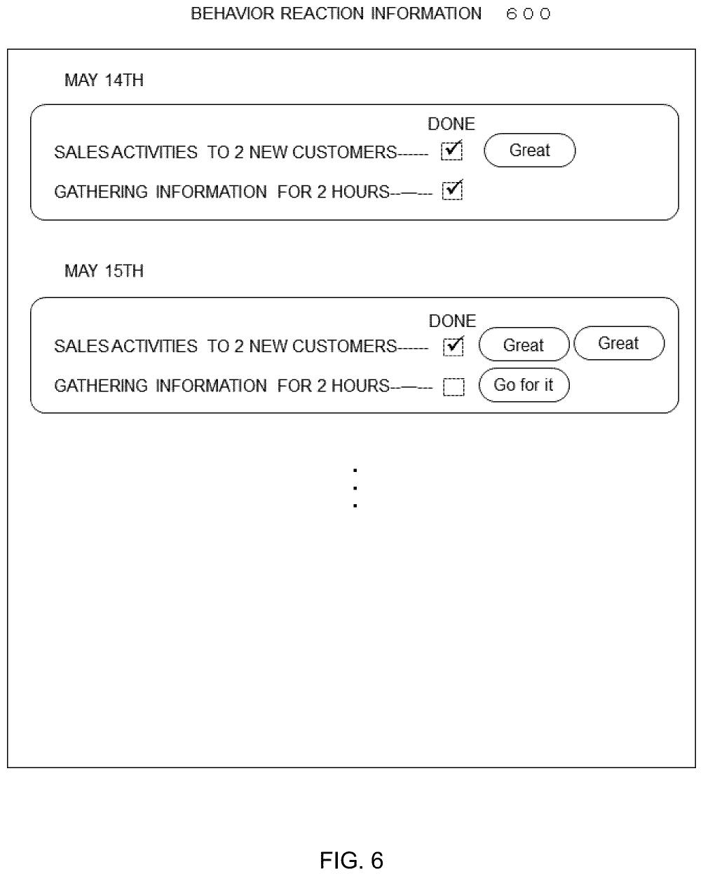

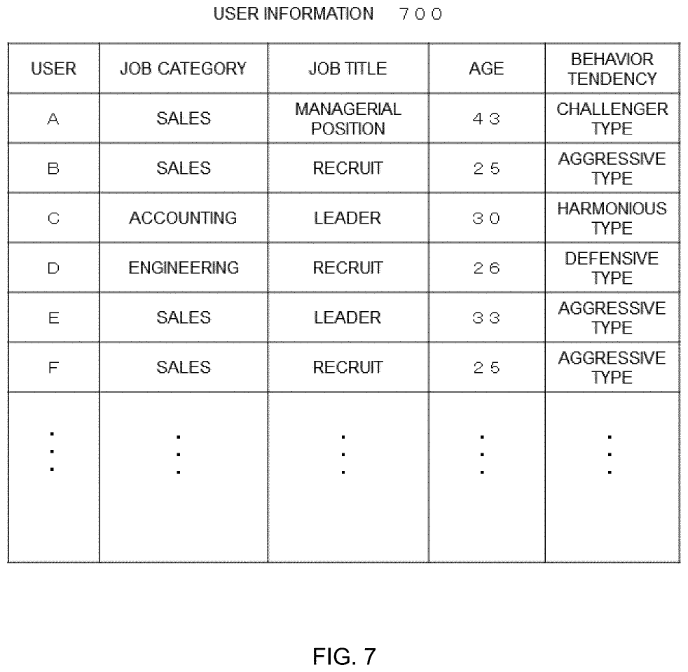

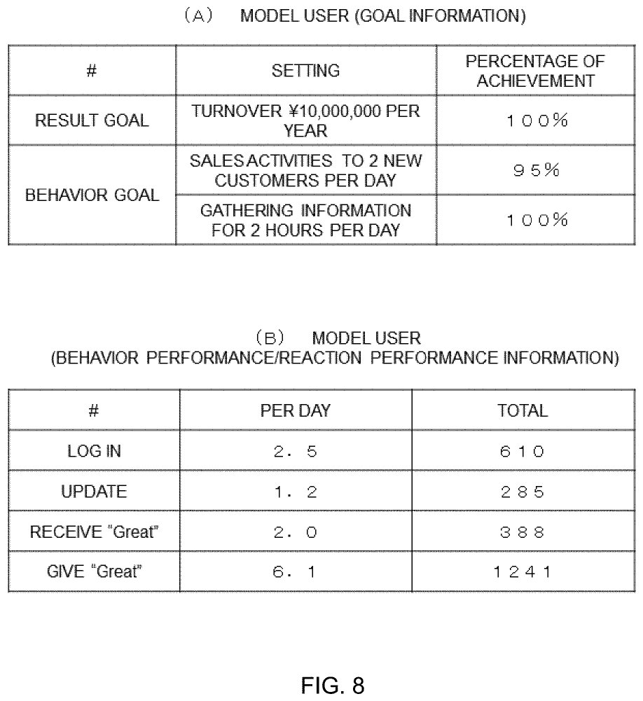

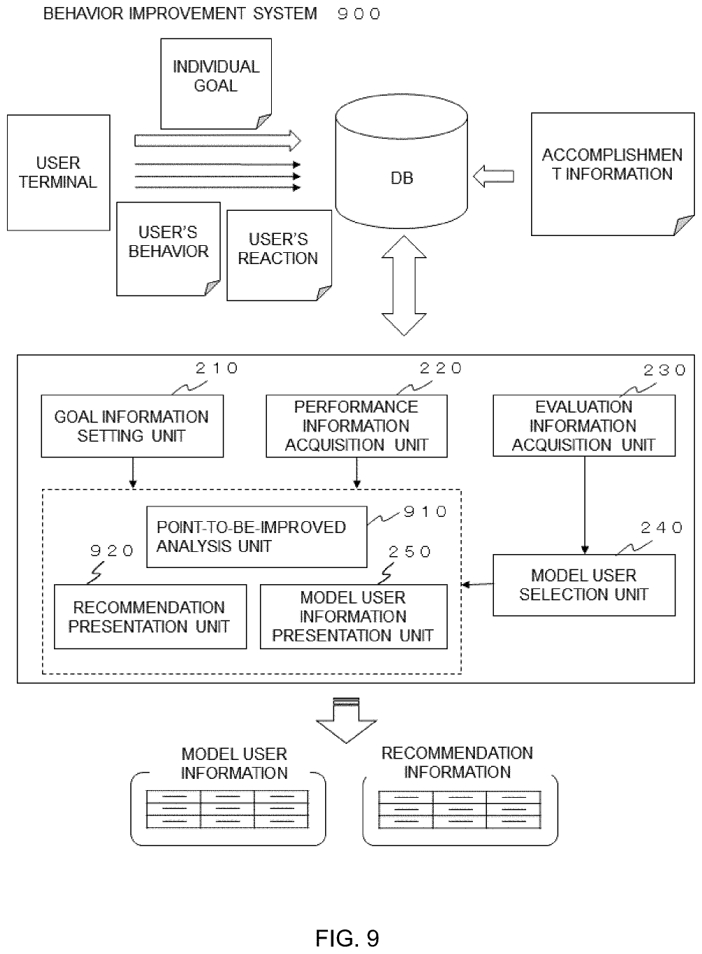

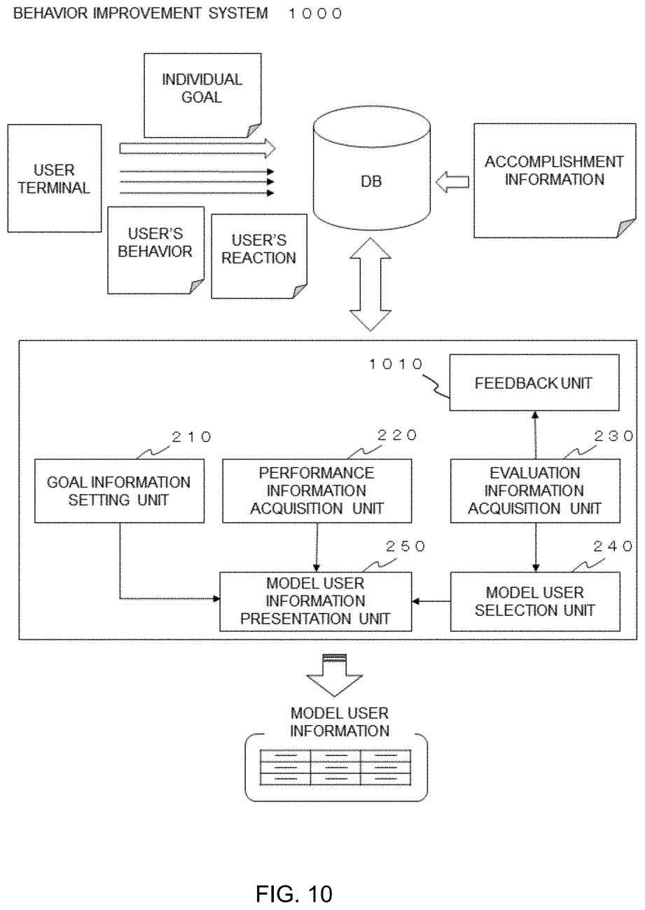

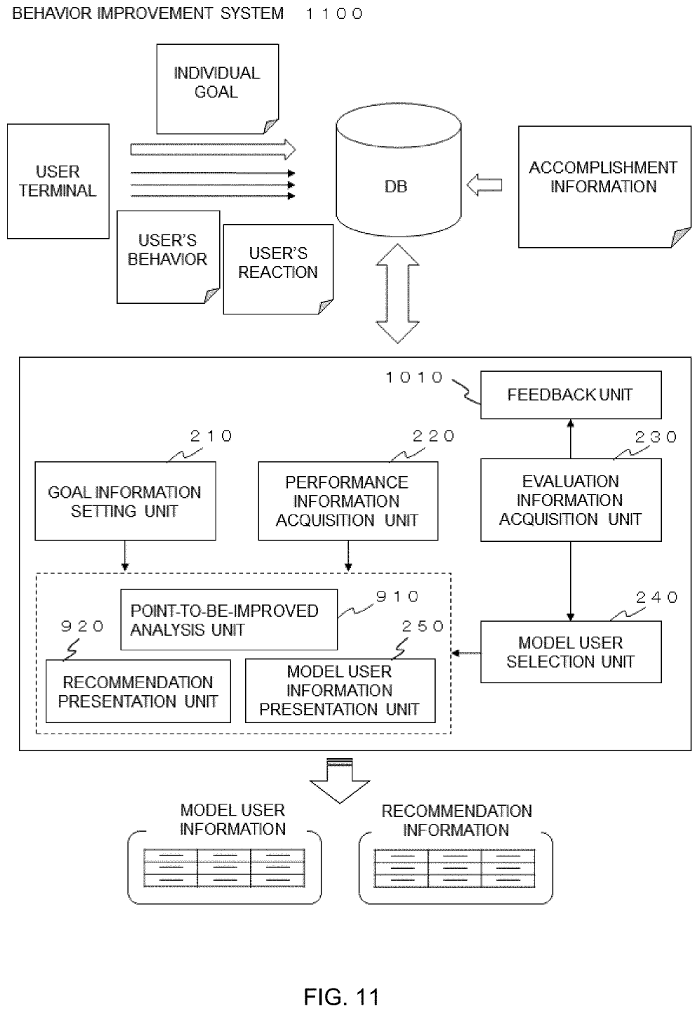

### BRIEF DESCRIPTION OF THE DRAWINGS

[FIG. 1] A diagram showing an outline of a system configuration according to the present invention.

[FIG. 2] A function block diagram showing functions of a behavior improvement system 200 according to a first embodiment of the present invention.

[FIG. 3] A flowchart showing a behavior improvement method 300 which is executed by the behavior improvement system 200 according to the first embodiment of the present invention.

[FIG. 4] A diagram showing goal information 400 relating to individual goals each of which is set by each of users.

[FIG. 5] A diagram showing behavior performance information 500 relating to user's behaviors toward behavior goals that are set in the goal information 400.

[FIG. 6] A diagram showing reaction performance information 600 relating to reactions that another user gives to the behavior performance information 500 relating to user's behaviors.

[FIG. 7] A diagram showing user information 700 which is information on each of users registered in the behavior improvement system 200 according to the first embodiment of the present invention.

[FIG. 8] A diagram showing: (A) goal information on a model user who serves as a goal among two or more users registered in the behavior improvement system 200 according to the first embodiment of the present invention; and (B) behavior performance information/reaction performance information on the model user.

[FIG. 9] A function block diagram showing functions of a behavior improvement system 900 according to a second embodiment of the present invention.

[FIG. 10] A function block diagram showing functions of a behavior improvement system 1000 according to a third embodiment of the present invention.

[FIG. 11] A function block diagram showing functions of a behavior improvement system 1100 which is identical to the behavior improvement system 900 according to the second embodiment of the present invention except that the behavior improvement system 1100 additionally includes a feedback unit 1010.

### DETAILED DESCRIPTION OF THE INVENTION

In the following, embodiments of the present invention will be specifically described with reference to the drawings. The embodiments below are shown just as specific exemplifications for carrying out the present invention, and should not be interpreted as limiting the present invention.

### First Embodiment

FIG. 1 outlines a system configuration of the present invention. Referring to FIG. 1, a system includes a server, a database (DB), and terminals (e.g., personal computers, smartphones, and the like) that are respectively operated by two or more users.

The server and terminals are connected via Internet, for example, and a network is made up of two or more users. The network, which is constituted on a company, department, workplace, district, school, tutoring school, class, project, club, circle, hobby, or generation basis for example, corresponds to a so-called social networking service (SNS) which is prevailing in recent years.

A network system is built that allows mutual information exchange in an organization a made up of users A to E, and allows mutual information exchange in an organization b made up of users F to H. Each user is able to participate in a network where he/she belongs by using an ID and/or a password.

To achieve such a network system, for example, programs and applications are allowed to run on the server and clients, and typically, the system is built as a server-client system.

FIG. 2 is a function block diagram showing functions of a behavior improvement system 200 according to a first embodiment of the present invention. FIG. 3 is a flowchart showing a behavior improvement method 300 which is executed by the behavior improvement system 200 according to the first embodiment of the present invention.

Referring to FIG. 2, the behavior improvement system 200 includes a goal information setting unit 210, a performance information acquisition unit 220, an evaluation information acquisition unit 230, a model user selection unit 240, and a model user information presentation unit 250. Referring to FIG. 3, the behavior improvement method 300 includes a goal information setting step S310, a performance information acquisition step S320, an evaluation information acquisition step S330, a model user selection step S340, and a model user information presentation step S350.

[Goal Information Setting Step]

In the goal information setting step S310, the goal information setting unit 210 sets goal information for each user, the goal information relating to an individual goal of the user. Here, the goal information includes information relating to a result goal which requires a predetermined period before being achieved and information relating to a behavior goal which is necessary in achieving the result goal.

More specifically, for example, when the behavior improvement system 200 is installed in a workplace, each user (employee) sets an annual individual goal as a result goal. The user deliberates on what behavior is needed to achieve the result goal, and sets the behavior as a behavior goal.

FIG. 4 is a diagram showing goal information 400 relating to individual goals each of which is set by each of users. In FIG. 4, a user A sets an annual turnover of ten million yen as a result goal, and sets sales activities to two new customers per day and gathering information for two hours per day as behavior goals which are behaviors needed to achieve the result goal. For users B and C as well, result goals and behavior goals are respectively set as shown in FIG. 4.

The goal setting is typically but not necessarily made from each user terminal at a timing when a new year begins, a new period begins, a new school term begins, a project is started, a study is started, or the like. The goal setting may be made as appropriate depending on the purpose of using the behavior improvement system 200 and situations of users.

Thanks to the information relating to the result goal which requires a predetermined period before being achieved and the information relating to the behavior goal which is necessary in achieving the result goal, the user is able to have the nearest goal definite and close to himself/herself.

[Performance Information Acquisition Step S320]

In the performance information acquisition step S320, the performance information acquisition unit 220 acquires behavior performance information and reaction performance information that are inputted by a user. The behavior performance information refers to information relating to user's behaviors toward individual goals that were set in the goal information setting step S310. For example, each user inputs his/her behavior as occasion arises, and such inputted information is recorded on a recording unit such as a memory, a DB, or the like.

FIG. 5 is a diagram showing behavior performance information 500 relating to user's behaviors toward behavior goals that are set in the goal information 400. For instance, assume a case where, in the goal information setting step S310, the user A sets an annual turnover of ten million yen as a result goal, and sets sales activities to two new customers per day and gathering information for two hours per day as behavior goals (see FIG. 4). In this case, the user A inputs whether or not the behavior goals that were set by himself/herself have been carried out. On May 14th, the sales activities to two new customers and the gathering information for two hours are checked as having been carried out, and on May 15th, only the sales activities to two new customers is checked as having been carried out.

In the above-described case, input of the behavior performance information is implemented by means of checking whether or not behavior goals that were set have been carried out. How to input, however, is not limited to this. For example, input of the behavior performance information may be implemented by means of inputting the degree of achievement of the behavior goals that were set. The behavior goals that were set by the user A will now be taken as an example. When behavior performance toward the behavior goal of sales activities to two new customers resulted in sales activities to zero new customer, “0” is inputted, when behavior performance resulted in sales activities to one new customer, “1” is inputted, and when behavior performance resulted in sales activities to two new customers, “2” is inputted. Additionally, displaying 0%, 50%, or 100% depending on the user's input may be acceptable, or displaying a graph or the like may also be acceptable.

In this example, the user A sets behavior goals on a one-day basis (behaviors to be carried out in a day). Typically, therefore, the behavior performance information is inputted once a day, and the input is repeated every day. It however is not always necessary that input is made once a day. The behavior performance information for few days may be inputted once every few days. Alternatively, input may be made twice a day in accordance with the number of behavior goal items. In a case of making no input, a status of “no input” or “no log-in” may be provided in addition to the information relating to whether or not goals are carried out.

In the example illustrated herein, the behavior performance toward the behavior goals are shown. Alternatively, it may be acceptable that performance toward result goals and/or the degree of achievement of the result goals is/are inputted as behavior performance. This may make an input operation troublesome for a user, but enables the user to recognize the performance in more detail and to recognize a position relative to the goal, which leads to an increased motivation of the user.

Reaction performance information will now be described. The reaction performance information refers to information relating to other users' reactions to behavior performance that a user inputs in the performance information acquisition step S320. For example, each user inputs his/her reaction as occasion arises, and such inputted information is recorded on a recording unit such as a memory, a DB, or the like.

FIG. 6 is a diagram showing reaction performance information 600 relating to reactions that another user gives to the behavior performance information 500 relating to user's behaviors. For example, as described above with FIG. 4, the user A carried out the sales activities to two new customers and the gathering information for two hours on May 14th, and carried out only the sales activities to two new customers on May 15th. To these behaviors, another user different from the user A gives reactions indicating “Great” and “Go for it”, thereby approving and encouraging the behaviors of the user A.

The reactions indicating “Great” and “Go for it” are not always given from only one user, but may be given from each of two or more users.

Reaction information is not limited to “Great” and “Go for it”. For example, other messages, stamps, and pictographs (emoji) may be used, or a scoring point or the like may be used.

The behavior performance information and the reaction performance information, which are inputted or updated in the performance information acquisition step S320 as described above, are preferably set such that, upon input or update made by a user, the input or update is notified to other users included in the network (organization). Other users' prompt reactions to the user's input could enable reflection of appropriate reaction performance information according to a situation at that time.

With the action of giving reactions expressed by “Great” and “Go for it”, the degree of interest in other users can be measured. In addition, as an expected effect, the action can contribute to an increased motivation of a user who has been given (who has received) the reaction information.

[Evaluation Information Acquisition Step]

In the evaluation information acquisition step S330, the evaluation information acquisition unit 230 acquires evaluation information on a user. The evaluation information refers to information that objectively indicates an evaluation on the user. In this embodiment, the evaluation information is, for example, accomplishment information indicating a user's accomplishment, which is obtained from a personnel division.

[Model User Selection Step]

In the model user selection step S340, the model user selection unit 240 selects a model user, who serves as a goal, from two or more users based on the evaluation information acquired in the evaluation information acquisition step S330. More specifically, the model user is a person having a high accomplishment. An ideal user that can be a goal of the subject user, and preferably a user who is familiar to the subject user and is in the same circumstances as the subject user is in, is selected as the model user.

FIG. 7 is a diagram showing user information 700 which is information on each of users registered in the behavior improvement system 200 according to the first embodiment of the present invention. Referring to FIG. 7, for example, to select a model user of the user A, a person having a high accomplishment may be selected as the model user from users in sales positions, a person having a high accomplishment may be selected as the model user from users in managerial positions, or alternatively a person having a high accomplishment may be selected as the model user from users in both of sales and managerial positions.

The model user is selected from users who belong to the same group as the subject user does and who are familiar to the subject user and are in the same circumstances as the subject user is in. Thus, following the model user as a role model seems to be realistic.

[Model User Information Presentation Step]

In the model user information presentation step S350, the model user information presentation unit 250 presents goal information, behavior performance information, and reaction performance information on the model user selected in the model user selection step S340.

FIG. 8 is a diagram showing: (A) goal information on a model user who serves as a goal among two or more users registered in the behavior improvement system 200 according to the first embodiment of the present invention; and (B) behavior performance information/reaction performance information on the model user. In a case where, for example, a user having the highest accomplishment in sales is selected as a model user in the model user selection step S340, goal information and behavior performance/reaction performance information on this user are presented as shown in FIG. 8.

In this case, peresented are the model user's result goal, behavior goals, the percentages of achieving them, the log-in count, the number of times the performance information has been updated, the number of times the model user has received “Great”, and the number of times the model user has given “Great”. A user, who recognizes the goal information and the behavior performance/reaction performance information on the model user thus presented, can consider working with an aim at the model user as a role model.

This system has a system configuration corresponding to a so-called SNS, and thus it may be possible that a user is able to post comments and/or photographs about daily lives and events, or to share articles or others' posts. These pieces of information and an aggregate of the pices (e.g., the number of comments, the number of posts, etc.) may be presented as information on the model user, too.

The user may work on future behavior improvements, by referring to setting of model user's result goal and behavior goal in relation to the result goal, and comparing them against setting of his/her result goal and behavior goal. For instance, the user sees whether the result goal and behavior goal are set concretely or abstractly, whether they are set in detail, or whether they are idealized or realistic.

The user may work on future behavior improvements, by referring to the frequency of model user's logging in, the frequency of model user's updating the performance information, the number of model user's receiving “Great”, and the number of model user's giving “Great”, and comparing them against his/her behavior performance and reaction performance. For instance, the user sees whether the model user frequently logs in, whether the model user frequently updates the behavior performance information, whether the model user frequently gives reaction information indicating a reaction such as “Great” to another member's performance, or how many “Greats” the model user has received from other members.

As mentioned above, the model user is selected from users who are familiar to the subject user and are in the same circumstances as the subject user is in. This is why the goal information and behavior performance/reaction performance information on the model user are very likely to be a role model, and the subject user is able to work on ideal and realistic behavior improvements.

Typically, goal information and behavior performance/reaction performance information on a single model user selected in the model user selection step S340 are presented. In the model user selection step S340, however, two or more model users may be selected. In such a case, information on the two or more model users may be presented. As the model user, a person having a high accomplishment is selected from users who are in the same circumstances as the subject user is in. For instance, a person having a high accomplishment is selected from persons belonging to a different job category from that of the subject user and having the same job title as that of the subject user, a person having a high accomplishment is selected from persons belonging to the same job category as that of the subject user and having a different job title from that of the subject user, and a person having a high accomplishment is selected from persons having the same behavior tendency as that of the subject user. Then, goal information and behavior performance/reaction performance information on these model users selected are presented.

It may be possible to present goal information and behavior performance/reaction performance information on the subject user as well as those on the model user. This makes comparison between the subject user's information and the model user's information easy, and allows differences between them to be recognized clearly. Such differences may be analyzed and presented in the behavior improvement system 200.

As thus far described, the behavior improvement system 200 and the behavior improvement method 300 according to the first embodiment of the present invention are configured to present the performance information on the model user serving as a goal, and thereby allow each user to autonomously work on behavior improvements without relying on a diagnostician, an advisor, and the like.

In this embodiment, user's job category, job title, age, individual goal, and behavior tendency are exemplified as the groups in the user information 700. This, however, is not limitative. For example, user's sex, department, job grade, when the user joined the company, background, past accomplishment, and the like may also be adoptable. The groups may be set as appropriate depending on users who use the behavior improvement system 200 and the behavior improvement method 300, situations, and environments.

In this embodiment, accomplishment information is exemplified as the evaluation information. This, however, is not limitative. For example, stress information indicating a user's stress condition may be adopted. Selecting a model user based on objective stress information allows a user to work on behavior improvements in relation to an appropriate stress condition.

In this embodiment, the behavior improvement system 200 and the behavior improvement method 300 are described as being used typically in a workplace, etc. This, however, is not limitative. For example, they may be used in the educational field including schools, tutoring schools, preparatory schools, and the like, or alternatively may be used in athletic and cultural clubs, baseball teama, football teams, sport clubs, golf circles, and the like.

For use in the educational field, for example, passing the entrance exam for the school a student wants to go to, a scoring point of a test, or the like is set as a result goal, while study hours per day, a progress on a textbook, or the like is set as a behavior goal. A user (senior) having passed into the school or a person having a high accomplishment in the test is selected as a model user. In this case, each user can autonomously work on behavior improvements by aiming at the model user as a role model.

### Second Embodiment

A behavior improvement system which will now be described as a second embodiment of the present invention is identical to the behavior improvement system 200 of the first embodiment except that a point to be improved is analyzed and a recommendation is presented.

FIG. 9 is a function block diagram showing functions of a behavior improvement system 900 according to the second embodiment of the present invention. In FIG. 9, elements identical to those of the behavior improvement system 200 according to the first embodiment of the present invention shown in FIG. 2 are denoted by the same corresponding reference signs, without detailed descriptions thereof. In this embodiment, elements different from the first embodiment of the present invention will be mainly described.

Referring to FIG. 9, the behavior improvement system 900 includes a goal information setting unit 210, a performance information acquisition unit 220, an evaluation information acquisition unit 230, a model user selection unit 240, a model user information presentation unit 250, a point-to-be-improved analysis unit 910, and a recommendation presentation unit 920.

The point-to-be-improved analysis unit 910 analyzes a point to be improved by the user. More specifically, the point-to-be-improved analysis unit 910 compares a subject user and a model user selected by the model user selection unit 240 in terms of goal information, behavior performance information, and reaction performance information, and analyzes differences therebetween.

In an example case, although the job category and job title of the subject user are the same as those of the model user, the subject user sets a lower goal, the subject user's behavior goal is less specific, the frequency of subject user's logging in is lower, or the number of “Greats” the subject user has given is smaller as compared to the model user. Such influences on the accomplishment information are analyzed. This analysis may be preliminarily set based on rules. The rules may be updated in accordance with data accumulation, or may be updated by using Artificial Intelligence (AI).

The recommendation presentation unit 920 presents a recommendation in accordance with the point to be improved that has been analyzed by the point-to-be-improved analysis unit 910. For instance, to a user having a low percentage of achieving the behavior goal, a message of “Keep at it every day” is presented, while to a user having a low log-in frequency, a message of “Log in more” is presented. To a user having a considerably low percentage of achieving the result goal, a message for recommending a review of the goal setting may be presented, or goal information on another user may be presented for reference.

The recommendation presentation unit 920 may present not only a message for bringing about behavior improvements but also a message for increasing a user's motivation, such as a message that evaluates the user's behaviors or a message that stirs up the user. These messages may be preliminarily set in association with the percentage of achieving the user's goal, a difference between the user and the model user, the user's job title, the user's job category, the user's age, the user's behavior tendency, or the like. Alternatively, these messages may be updated in accordance with data accumulation, or may be updated by using AI.

As thus far described, the behavior improvement system 900 according to the second embodiment of the present invention includes the point-to-be-improved analysis unit 910 and the recommendation presentation unit 920 in addition to the elements included in the behavior improvement system 200 according to the first embodiment. This allows each user to autonomously work on behavior improvements without relying on a diagnostician, an advisor, and the like.

### Third Embodiment

A behavior improvement system which will now be described as a third embodiment of the present invention is identical to the behavior improvement system 200 of the first embodiment except that a result of each user's working on behavior improvements by aiming at a model user as a role model is fed back.

FIG. 10 is a function block diagram showing functions of a behavior improvement system 1000 according to the third embodiment of the present invention. In FIG. 10, elements identical to those of the behavior improvement system 200 according to the first embodiment of the present invention shown in FIG. 2 are denoted by the same corresponding reference signs, without detailed descriptions thereof. In this embodiment, elements different from the first embodiment of the present invention will be mainly described.

Referring to FIG. 10, the behavior improvement system 900 includes a goal information setting unit 210, a performance information acquisition unit 220, an evaluation information acquisition unit 230, a model user selection unit 240, a model user information presentation unit 250, and a feedback unit 1010.

To a user, the model user information presentation unit 250 presents model user's goal information, behavior performance information, and reaction performance information. The user refers to the model user's goal information, behavior performance information, and reaction performance information that are presented, to change his/her behavior goal and/or result goal, or to improve his/her behavior and/or reaction. This consequently brings a change of evaluation information before and after the presentation of the model user's goal information, behavior performance information, and reaction performance information.

The feedback unit 1010 feeds back the change of evaluation information before and after the presentation of the model user's goal information, behavior performance information, and reaction performance information. For example, if the accomplishment is ascending, it is determined that an effect is obtained from the presentation of the model user's goal information, behavior performance information, and reaction performance information, and additionally it is recorded that the model user selection for the subject user has been appropriate. If the accomplishment is not changing or is descending, it is determined that no effect is obtained from the presentation of the model user's goal information, behavior performance information, and reaction performance information, and it is recorded that the model user selection for the subject user has not been appropriate.

In this manner, a change of evaluation information before and after the presentation of the model user's goal information, behavior performance information, and reaction performance information is fed back, and whether or not the model user selection has been appropriate is accumulated, which can be utilized for futher model user selection. The model user selection unit 240 selects a model user serving as a goal from two or more users based on the evaluation information. At that time, making the selection based on the data accumulated by feedback enables the model user selection unit 240 to select a more appropriate model user.

Not only a change of evaluation information such as the accomplishment but also, for example, a change of goal information or performance information may be fed back. Even when the evaluation information such as the accomplishment is not changing, whether or not daily behaviors are changing may be determined so that whether or not the motivation is increasing can be determined.

Such information may be analyzed by using AI, for selection of a model user.

Although the configuration described herein has the feedback unit 1010 in addition to the behavior improvement system 200 of the first embodiment, a configuration having the feedback unit 1010 in addition to the behavior improvement system 900 of the second embodiment may be acceptable, too. FIG. 11 is a function block diagram showing functions of a behavior improvement system 1100 which is identical to the behavior improvement system 900 according to the second embodiment of the present invention except that the behavior improvement system 1100 additionally includes the feedback unit 1010. The behavior improvement system 1100 is identical to the behavior improvement system 1000 except that the behavior improvement system 1100 additionally has a configuration of feeding back whether or not presentation of recommendation information has been appropriate.

More specifically, to a user, the model user information presentation unit 250 presents model user's goal information, behavior performance information, and reaction performance information, and the recommendation presentation unit 920 presents a recommendation. The user refers to the model user's goal information, behavior performance information, and reaction performance information as well as the recommendation that are presented, to change his/her behavior goal and/or result goal, or to improve his/her behavior and/or reaction. This consequently brings a change of evaluation information before and after the presentation of the model user's goal information, behavior performance information, and reaction performance information as well as the recommendation.

The feedback unit 1010 feeds back the change of evaluation information before and after the presentation of the model user's goal information, behavior performance information, and reaction performance information as well as the recommendation. For example, if the accomplishment is ascending, it is determined that an effect is obtained from the presentation of the model user's goal information, behavior performance information, and reaction performance information as well as the recommendation. In addition, it is recorded that the model user selection for the subject user has been appropriate, or it is recorded that the recommendation has been appropriate. If the accomplishment is not changing or is descending, it is determined that no effect is obtained from the presentation of the model user's goal information, behavior performance information, and reaction performance information as well as the recommendation, and it is recorded that the model user selection for the subject user has not been appropriate and/or that the recommendation has not been appropriate.

In this manner, a change of evaluation information before and after the presentation of the model user's goal information, behavior performance information, and reaction performance information as well as the recommendation is fed back, and whether or not the model user selection and/or the recommendation has/have been appropriate is accumulated, which can be utilized for future model user selection and/or recommendation. The model user selection unit 240 selects a model user serving as a goal from two or more users based on the evaluation information. At that time, making the selection based on the data accumulated by feedback enables the model user selection unit 240 to select a more appropriate model user. Likewise, selecting a recommendation based on the data accumulated by feedback enables the recommendation presentation unit 920 to select a more appropriate recommendation.

### Other Embodiments

The behavior improvement system 200 according to the first embodiment of the present invention, the behavior improvement system 900 according to the second embodiment of the present invention, and the behavior improvement system 1000 and behavior improvement system 1100 according to the third embodiment of the present invention described above may include a correlation analysis unit, a motivation information acquisition unit, and a health information acquisition unit.

The correlation analysis unit analyzes a correlation of the behavior performance information and reaction performance information that are acquired by the performance information acquisition unit 220 with the evaluation information that is acquired by the evaluation information acquisition unit 230. Typically, the behavior performance information and reaction performance information are correlated with the evaluation information. Analyzing the correlation and making a user work on behavior improvements contribute to efficiency.

To analyze a correlation, it is just required that patterns that can be expected are preliminarily set or analyzed by using AI. Since a factor in an objective result can be grasped, each user can autonomously work on behavior improvements in a more appropriate manner.

The motivation information acquisition unit provides a questionnaire to a user at a predetermined timing, and based on a result of the user's answering the questionnaire, acquires motivation information which indicates the user's psychological state.

A user's motivation changes from day to day, and as a matter of course, goes up and down. The user's motivation may be naturally influenced by, for example, a private life which has nothing to do with the workplace, etc. The user's motivation, however, is also correlated with the degree of achievement of a result goal and a behavior goal, reception of “Great” from another user, or giving of “Great”.

Herein, a questionnaire is provided to a user so that motivation information on the user is acquired when the user sets an individual goal, when the user inputs behavior performance information and reaction performance information, or for every fixed period. The correlation analysis unit analyzes a correlation among the behavior performance information, the reaction performance information, the evaluation information, and the motivation information.

For example, it is normally expected that as the degree of achievement of the behavior goal is low, the motivation tends to be lowered, too. Here, assume that the model user increases his/her motivation by repeatedly receiving and giving “Greats” from and to other users. If this correlation can be derived, it is possible to present the above-described recommendation with the motivation taken into account.

The health information acquisition unit acquires health information indicating a user's health state. The health information may be acquired based on a result of the user's answering a questionnaire that has been provided to the user at a predetermined timing, or alternatively may be acquired based on a result of a health diagnosis.

The user's health state, like the motivation described above, changes from day to day, and as a matter of course, goes up and down. Analyzing a correlation of the degree of achievement of a result goal and a behavior goal with a daily health state may result in a discovery that, for example, a user having a high degree of achievement or a person having a high accomplishment has a stable health state. In this case, a recommendation for paying attention to a health state can be presented to each user.

It is preferable that each pieses of information analyzed by the correlation analysis unit described herein is analyzed especially in accordance with a behavior tendency shown in FIG. 7. A behavior improvement that is optimum for a user is likely to rely on the user's behavior tendency. Therefore, a recommendation is presented so as to recommend behavior improvements not only based on objective result information but also in accordance with each individual user.

For example, a user of aggressive type tends to set a high result goal and a high behavior goal, which results in a low percentage of achievement. In such a case, a determination is made in consideration of the user's behavior tendency, instead of simply determining that the percentage of achievement is low.

In providing a recommendation, a recommendation that is optimum to a user is provided in accordance with the user's behavior tendency, for example, in consideration of the possibility of humiliating the user or the necessity to put excessive pressure on the user.

For making the determination and providing the recommendation, a lot of data is accumulated, a variety of patterns are preliminarily set, and the variety of patterns are updated with AI learning.

While the embodiments of the present invention have been described in terms of behavior improvements of an individual user, the behavior improvement system and behavior improvement method according to the present invention are applicable to an organization or a group (e.g., a department, a division, a project team, or the like, in a company) including two or more users. To be specific, a group goal is set, a model group that is superior in accomplishment is selected, and information on the model group is presented. Behavior performance information and reaction performance information that are presented in this case may be behavior performance information and reaction performance information on each of users included in the group, or may be a result of gathering and analyzing behavior performance information and reaction performance information on each of users included in the group.

It may be also acceptable to analyze a correlation between the accomplishment and attributes of users included in the group that is superior in accomplishment. Examples of the attributes include whether users' behavior tendencies are biased or divergent, the male-to-female ratio, and the balance of age groups or job titles. A recommendation may be presented based on a result of the analysis.

While some embodiments of the present invention have been specifically described above, those embodiments are only exemplifications. The scope of the present invention is not limited to those embodiments, but should be construed broadly to encompass a range comprehensible by those skilled in the art.

### INDUSTRIAL APPLICABILITY

The present invention is applicable to, for example, a behavior improvement system that prompts a user to work on behavior improvements, and especially useful to organizations and groups in workplaces of companies or public offices, in the educational field including schools, tutoring schools, and preparatory schools, in clubs and circles, and the like.

### DESCRIPTION OF THE REFERENCE NUMBERS

200, 900, 1000, 1100 behavior improvement system

210 goal information setting unit

220 performance information acquisition unit

230 evaluation information acquisition unit

240 model user selection unit

250 model user information presentation unit

300 behavior improvement method

S310 goal information setting step

S320 performance information acquisition step

S330 evaluation information acquisition step

S340 model user selection step

S350 model user information presentation step

400 goal information

500 behavior performance information

600 behavior reaction information

700 user information

910 point-to-be-improved analysis unit

920 recommendation presentation unit

1010 feedback unit

1. A behavior improvement system that prompts a user to work on behavior improvements by using a network system that enables information exchange in an organization including two or more users, the behavior improvement system comprising:
a goal information setting unit that sets goal information relating to an individual goal of the user;
a performance information acquisition unit that acquires behavior performance information and reaction performance information inputted by the user, the behavior performance information relating to a behavior of the user toward the individual goal, the reaction performance information relating to a reaction of the user to a behavior of another user different from the user;
an evaluation information acquisition unit that acquires evaluation information on the user;
a model user selection unit that selects a model user serving as a goal from the two or more users based on evaluation information acquired by the evaluation information acquisition unit; and
a model user information presentation unit that presents goal information, behavior performance information, and reaction performance information on the model user selected by the model user selection unit.

2. The behavior improvement system according to claim 1, wherein
the goal information includes information relating to a result goal that requires a predetermined period before being achieved and information relating to a behavior goal that is necessary in achieving the result goal.

3. The behavior improvement system according to claim 2, wherein
the behavior performance information includes information relating to the degree of achievement of the result goal and behavior goal.

4. The behavior improvement system according to claim 1, wherein
the reaction performance information includes information relating to approving and/or encouraging a behavior of another user different from the user.

5. The behavior improvement system according to claim 1, further comprising a feedback unit that feeds back evaluation information acquired by the evaluation information acquisition unit after the goal information, behavior performance information, and reaction performance information on the model user are presented by the model user information presentation unit.

6. The behavior improvement system according to claim 1, wherein
the model user selection unit selects the model user from ones of the two or more users belonging to the same group as the user does.

7. The behavior improvement system according to claim 6, wherein
the group is constituted based on commonness of users in terms of at least any of a job category, a job title, an age, an individual goal, or a behavior tendency.

8. The behavior improvement system according to claim 1, wherein
the evaluation information is accomplishment information indicating an accomplishment of the user.

9. The behavior improvement system according to claim 1, wherein
the evaluation information is stress information indicating a stress condition of the user.

10. The behavior improvement system according to claim 1, further comprising:
a point-to-be-improved analysis unit that compares the goal information, behavior performance information, and reaction performance information on the user against the goal information, behavior performance information, and reaction performance information on the model user, and analyzes a point to be improved in the individual goal of the user, the behavior of the user, the reaction of the user to a behavior of another user different from the user; and
a recommendation presentation unit that presents a recommendation in accordance with the point to be improved analyzed by the point-to-be-improved analysis unit.

11. The behavior improvement system according to claim 1, further comprising a correlation analysis unit that analyzes a correlation of behavior performance information and reaction performance information acquired by the performance information acquisition unit with evaluation information acquired by the evaluation information acquisition unit.

12. The behavior improvement system according to claim 11, further comprising a motivation information acquisition unit that provides a questionnaire to the user at a predetermined timing, and based on a result of the user's answering the questionnaire, acquires motivation information indicating a psychological state of the user, wherein
the correlation analysis unit analyzes a correlation among the behavior performance information, the reaction performance information, the evaluation information, and the motivation information.

13. The behavior improvement system according to claim 12, wherein
the predetermined timing comes when the user sets an individual goal, when the user inputs behavior performance information and reaction performance information, or for every fixed period.

14. The behavior improvement system according to claim 11, further comprising a health information acquisition unit that acquires health information indicating a health state of the user, wherein
the correlation analysis unit analyzes a correlation among the behavior performance information, the reaction performance information, the evaluation information, and the health information.

15. A behavior improvement method executed by a behavior improvement system that prompts a user to work on behavior improvements by using a network system that enables information exchange in an organization including two or more users, the method comprising:
a goal information setting step of setting goal information relating to an individual goal of the user;
a performance information acquisition step of acquiring behavior performance information and reaction performance information inputted by the user, the behavior performance information relating to a behavior of the user toward the individual goal, the reaction performance information relating to a reaction of the user to a behavior of another user different from the user;
an evaluation information acquisition step of acquiring evaluation information on the user;
a model user selection step of selecting a model user serving as a goal from the two or more users based on evaluation information acquired in the evaluation information acquisition step; and
a model user information presentation step of presenting goal information, behavior performance information, and reaction performance information on the model user selected in the model user selection step.
